(19) United States
(12) Patent Application Publication
(10) Pub. No.: US 2021/0025897 A1
(43) Pub. Date: Jan. 28, 2021

(54) METHOD FOR DETERMINING THE ANTIOXIDANT CAPACITY OF A BIOLOGICAL SAMPLE AND RELATED KIT

(71) Applicant: FONDAZIONE ISTITUTO ITALIANO DI TECNOLOGIA, Genova (IT)

(72) Inventors: Mauro MOGLIANETTI, Civitanova Marche (Macerata) (IT); Pier Paolo POMPA, Lecce (IT)

(21) Appl. No.: 16/495,200

(22) PCT Filed: Mar. 19, 2018
(86) PCT No.: PCT/IB2018/051813

§ 371 Date: Sep. 18, 2019

(30) Foreign Application Priority Data
Mar. 21, 2017 (IT) 102017000030715

(51) Int. Cl. G01N 33/58; G01N 21/33; G01N 33/02; G01N 31/22

(52) U.S. Cl. CPC G01N 33/587; G01N 21/33; G01N 33/02; G01N 31/22; G01N 2800/364; G01N 2800/042; G01N 2800/107; G01N 2800/347; G01N 2800/7009

(57) ABSTRACT

A method is provided for determining antioxidant power of a sample of a biological fluid or a food. The method essentially consists in contacting the sample to be tested with an aqueous solution of platinum nanoparticles, an oxidizing agent, and a chromogenic peroxidase substrate, and detecting color of the final solution thus obtained. Color intensity of the solution is proportional to the antioxidant power of the sample. A kit suitable for carrying out the method is also provided.

### CROSS-REFERENCE TO RELATED APPLICATIONS

This application is a National Phase filing of PCT International Application No. PCT/IB2018/051813, having an International Filing Date of Mar. 19, 2018, claiming priority to Italian Patent Application No. 102017000030715, having a filing date of Mar. 21, 2017 each of which is hereby incorporated by reference in its entirety.

### FIELD OF THE INVENTION

The present invention relates to a method and a kit for determining the antioxidant capacity of a biological sample.

### BACKGROUND OF THE INVENTION

The oxidative stress and the power of modulating it through antioxidants have attracted much attention in the scientific community, since numerous scientific studies and epidemiological investigations have linked oxidative stress to various diseases such as cancer, renal injury, gout, endometriosis, diabetes.

In this context, saliva and other biological fluids such as blood, sweat and urine have important diagnostic potential, reflecting the physiological state of the individual at the time of sample collection. In particular, saliva is known to contain molecules produced by the salivary glands together with serum components transported into the saliva by passive diffusion through the capillaries, as well as other material released by the cells.

To date, there is a large body of literature linking the antioxidative state of saliva and other biological fluids to the onset of serious pathologies. The oxidative stress values in saliva and blood have also been shown to be strongly correlated.

The scientific and medical community is therefore focusing on various salivary biomarkers that allow monitoring of the body condition, such as for example the Total Antioxidant Capacity (TAC). TAC is of great interest because it comprises all the components capable of modulating oxidation, including enzyme and non-enzyme molecules, thus representing the net antioxidant power. TAC modifications can be bidirectional and denote systemic or specific changes in the redox homeostasis of certain tissues.

At the same time, there is a growing interest in the role that antioxidants in food play in human health, given that the beneficial effect resulting from the consumption of fruit, vegetables, tea, coffee and cocoa has actually been attributed to antioxidants.

To date, the main methods for determining the antioxidant capacity of a sample of a biological fluid or a food, for clinical or food analysis, are based on the power of an antioxidant to reduce the free radicals generated through a synthetic procedure, reduce specific metal ions (for example, copper, gold, iron), block fluorogenic radical molecules or modify chromogenic substrates (for example: ABTS (2,2′-azino-bis(3-ethylbenzothiazoline-6-sulphonic acid), DPPH (1,1-diphenyl-2-picrylhydrazyl), TMB (3,3′,5,5′-tetramethylbenzidine), or block superoxide anions synthetically produced.

Much attention has been focused on the development of new tests capable of measuring the total antioxidant power of a given biological sample, in order to be able to assess the total antioxidant capacity thereof. The excessive variety of the available tests, however, has produced a lack of consistency among the results obtainable with the different systems, with a consequent lack of homogeneity and fragmentation of the results. Often, it is also necessary to resort to the use of multiple tests to assess the overall real antioxidant power of a sample.

There is also a lack of tests that lend themselves to be implemented through portable devices, point-of-care devices, and devices executable by the end user. A test with these characteristics would allow a continuous and effective monitoring of the antioxidant power of the organism through the main biological fluids.

An ideal standard method should possess essentially all of the following features:

measure the sample directly, without purification steps;

be simple and low cost;

have a clear physical-chemical mechanism of operation;

not require specific instrumentation;

be reproducible and reliable;

be stable in ambient conditions;

measure both lipophilic and hydrophilic antioxidants;

be usable for studies on large numbers of samples.

The methods developed so far fall into two main categories: Hydrogen Atom Transfer (HAT) and Single Electron Transfer (SAT). Methods belonging to the first category measure the ability of an antioxidant to block the free radicals through the donation of hydrogen. Instead, SAT methods measure the ability of an antioxidant to transfer an electron to reduce a compound, whether it is a metal, a carbonyl or a radical.

The most common HAT methods are:

Total radical-trapping antioxidant parameter (TRAP),

Oxygen radical absorbance capacity,

Inhibition of induced LDL oxidation,

Total oxyradical scavenging capacity assay (TOSCA).

The most common SET methods are:

Trolox equivalence antioxidant capacity (TEAC) assay,

Ferric ion reducing antioxidant power (FRAP) assay,

Cupric ions reducing antioxidant power (CUPRAC),

Total antioxidant potential assay with Cu-complex as oxidant,

2,2-Diphenyl-1-picrylhydrazyl radical (DPPH) scavenging,

2,2-Azinobis 3-ethylbenzthiazoline-6-sulphonic acid radical (ABTS) scavening assay,

N,N-dimethyl-p-phenylenediamine radical (DMPD) scavening assay,

In addition to the two categories mentioned above, there are also methods that selectively measure the ability of a given substance to block certain biologically relevant oxidants. The most important of these are: Hydrogen peroxide scavenging assay, Superoxide anion radical scavenging assay, and Hydroxyl radical scavenging assay.

New methods for determining the antioxidant power of fluids have been emerging in recent years and consist in the use of gold nanoparticles as a colorimetric sensor. These methods are based on the reduction of gold ions into nanoparticles with the consequent generation of color associated with the formation of metal nanoparticles. The property of antioxidants to increase the size of the gold nanoparticles and hence their optical properties has also been proposed as a method of detecting antioxidants.

Another method uses the inhibition of the formation of gold nanoparticles by hydrogen peroxide, a phenomenon that results in a lack of staining of the solution. A method also based on gold nanoparticles and their optical properties has further been proposed for detecting antioxidants, which is based on the clustering of colloidal gold nanoparticles and the consequent change in color of the solution.

A further known method for detecting antioxidants in food uses cerium nanoparticles and is based on the change in color of the nanomaterial upon contact with the food in the liquid phase (U.S. Pat. No. 8,969,085).

Some of the known methods for measuring the antioxidant power of a biological fluid or a food are based on redox reactions catalyzed by antioxidant enzymes (e.g. peroxidase and catalase). However, the latter have high isolation and purification costs, and are extremely sensitive to proteases, pH and temperature. Therefore, the scientific interest is to develop “artificial enzymes”, designated as nanozymes, including platinum nanoparticles, which are ideal for their efficient and selective catalase and peroxidase activities.

Platinum nanoparticles are obtained by simple and inexpensive synthesis and purification protocols, are stable, maintain the catalytic activity unchanged even under extreme temperature and pH conditions, and resist the action of proteases. They are also able to oxidize chromogenic peroxidase substrates (in particular 3,3′,5,5′-tetramethylbenzidine (TMB), 3,3′-diaminobenzidine (DAB)) in the presence of hydrogen peroxide as the oxidizing agent, with a higher affinity for TMB than biological peroxidase.

In recent years, several colorimetric tests based on the use of platinum nanoparticles have been developed for the detection of DNA, tumor cells, metal ions, penicillin-based antibiotics, hydrogen peroxide, glucose, cholesterol, L-cysteine, proteins and antibodies.

The first biological assay based on the use of platinum nanoparticles was proposed more than ten years ago by Gill, R. et al. Pt Nanoparticles Functionalized with Nucleic Acid Act as Catalytic Labels for the Chemiluminescent Detection of DNA and Proteins, Small 2, 1037-1041, doi:10.1002/smll.200600133 (2006), who developed a protein detection system based on the chemiluminescent reaction between luminol and hydrogen peroxide, catalyzed by complexes of platinum nanoparticles and aptamers. Instead, irregularly shaped platinum nanoparticles functionalized with anti-RIgG antibodies have been proposed as peroxidase substitutes in an enzyme-linked immunosorbent assay (ELISA) for the colorimetric determination of rabbit IgGs, using TMB and H₂O₂ as substrates (Gao, Z. et al. irregular-shaped platinum nanoparticles as peroxidase mimics for highly efficient colorimetric immunoassay, Analytica chimica acta 776, 79-86 (2013)). In the food sector, the TMB oxidation mechanism promoted by gold and platinum nanoparticles functionalized with 4-mercaptophenylboronic acid has been applied in the development of a “naked eye” biosensor for detecting Escherichia coli in food and water samples.

In the clinical field, instead, the TMB chromogen, associated with nanohybrid systems of magnetic Fe₃O₄ nanoparticles and platinum nanoparticles, was used immobilized on a graphene oxide surface, in order to colorimetrically identify breast cancer cells.

However, none of the currently available assays allows the antioxidant power to be assessed through a point-of-care, naked-eye determination, without the need of specific instrumentation.

### SUMMARY OF THE INVENTION

These and other needs are met by the present invention, which provides a method for determining the antioxidant power of a sample of a biological fluid or a food which, compared to the techniques described in the state of the art, requires very short times (about 5 minutes), is based on the inhibition of color development by antioxidants present in the sample itself, requires no pretreatment of the sample, and is extremely low cost. Moreover, the detection of the signal, i.e. the color, can be performed with the naked eye and therefore on the spot (“point-of-care”), without necessarily requiring the use of specific instrumentation.

The method of the invention is described and claimed herein. The scope of the invention also includes a kit suitable for implementing the method of the invention.

Further features and advantages are also described.

### BRIEF DESCRIPTION OF THE DRAWINGS

FIG. 1 shows results expressed as absorbance values measured at 652 nm obtained by performing the method of the invention on saliva samples from healthy volunteers;

FIG. 2 shows detection by the naked eye, using a reference color scale;

FIG. 3 shows results expressed as absorbance values measured at 652 nm obtained by performing the method of the invention on food samples.

### DETAILED DESCRIPTION

In the method of the present invention, the platinum nanoparticles perform a catalytic function. As an alternative to nanoparticles made entirely of platinum, platinum-based nanoparticles can be used in combination with another metal such as gold, palladium and/or silver.

The substrate of the oxidation reaction is preferably the chromogen TMB (3,3′,5,5′-tetramethylbenzidine). Hydrogen peroxide is typically used as the oxidizing agent.

The method of the invention is based on the fact that the antioxidant substances in the sample under examination interact with hydrogen peroxide, causing a partial or total inhibition of the TMB oxidation reaction. The result is a less intense development of the blue color, which, as is known, is formed by oxidation of the TMB chromogenic substrate. The observable decreased oxidation of TMB by hydrogen peroxide is directly proportional to the amount of antioxidant substances and can therefore be used to colorimetrically quantify the concentration of antioxidants in the sample under examination. This reaction scheme is particularly effective and specific since it allows the total antioxidant capacity of a sample to be measured without the need of any preliminary purification step.

The assay method of the present invention can be applied to several biological fluids such as saliva, blood, sweat, or urine. It can also be applied to liquid food such as fruit juices and edible oils. In the case of oils, it is preferable that the sample is first mixed with a solution of methanol and isopropanol, to which the above listed reagents are subsequently added, without however requiring separation or purification.

The examples that follow are provided for illustration purposes only and do not limit the scope of the invention as described and claimed herein.

### EXAMPLES

The following assay is performed:

400 microlitres of an acetate buffer solution, typically between 0.01 and 1 M, preferably between 0.05 and 0.3 M were added to a test tube; the pH can vary between 1 and 7, preferably between 3 and 5.5;

200 microlitres of a TMB 3,3′,5,5′-tetramethylbenzidine) solution were added at a concentration of between 0 and 1 M, preferably between 0.002 and 0.05 M;

100 microlitres of a solution containing platinum nanoparticles were added at a concentration comprised between 0.01 and 1000 ppm platinum, preferably between 0.1 and 10 ppm; the diameter of the nanoparticles may vary between 0.1 nm and 1000 nm, preferably between 1 and 100 nm;

100 microlitres of the sample to be tested, previously diluted in an aqueous solution by a factor comprised between 1:2 and 1:500, preferably between 1:2 and 1:100, were added; if the sample is in the form of an oil it is first mixed with a solution of methanol and isopropanol;

the color development reaction (blue) was initiated by the addition of 200 microliters of a 1 M hydrogen peroxide solution.

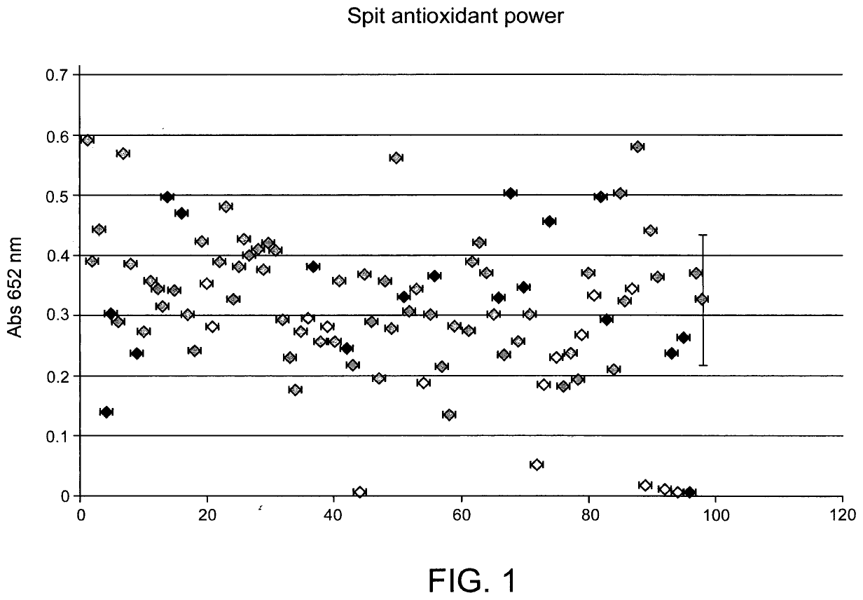

FIG. 1 shows the results obtained by performing the test on 100 saliva samples obtained from healthy volunteers and carrying out the detection by UV-Vis spectroscopy (measurement of the absorbance at 652 nm). Absorbance values lower than 0.1 correspond to saliva samples obtained following the intake of food supplements (Vitamin C).

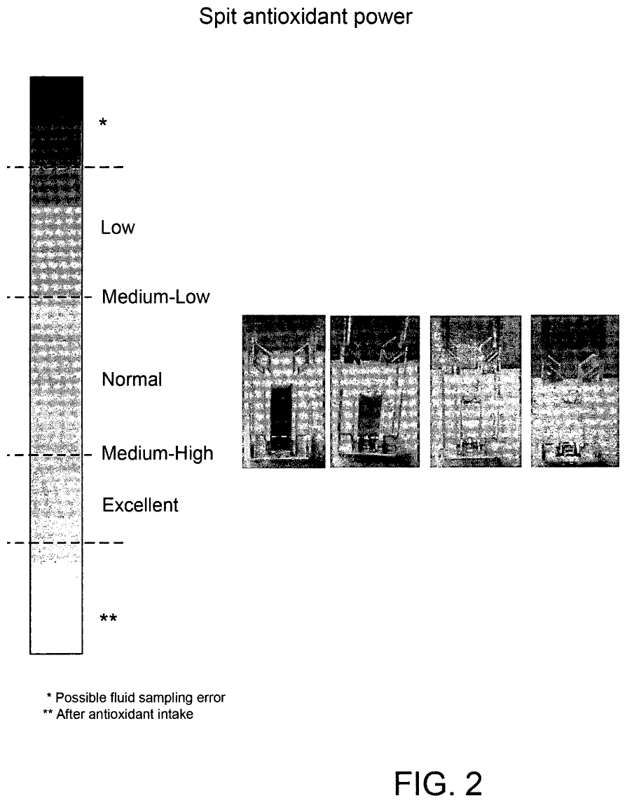

As an alternative to the spectroscopic method, it is possible to carry out the detection by the naked eye, using a reference color scale (which shows different color intensity bands and the related antioxidant score (e.g., “excellent”, “medium-high”, “standard”, “medium-low”, “low”, cf. FIG. 2).

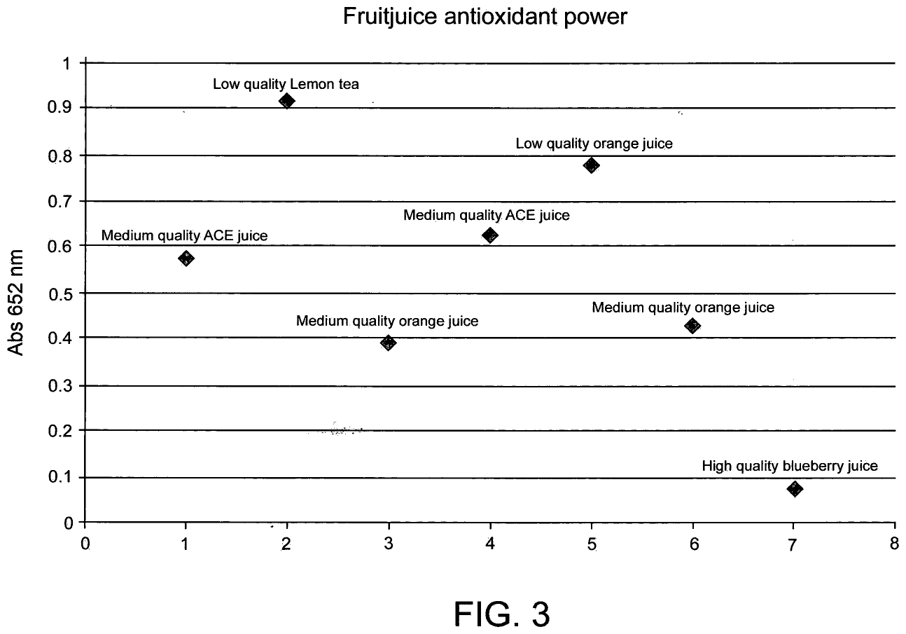

As indicated above, the method of the invention can also be used to analyze food samples such as fruit juices. FIG. 3 shows the results obtained by testing food samples of fruit juice and other industrial drinks and performing the detection by UV-Vis spectroscopy (measurement of the absorbance at 652 nm).

1. A method for determining antioxidant power of a sample of a biological fluid or a food, comprising the steps of:
contacting the sample with
an aqueous solution of metal nanoparticles, wherein said metal nanoparticles comprise platinum optionally in combination with gold, palladium and/or silver,
an oxidizing agent, and
a chromogenic peroxidase substrate, and
detecting color intensity of a final solution thereby obtained, the color intensity being proportional to the antioxidant power of the biological sample.

2. The method of claim 1, wherein the chromogenic peroxidase substrate is 3,3′,5,5′-tetramethylbenzidine (TMB).

3. The method of claim 1, wherein the oxidizing agent is hydrogen peroxide.

4. The method of claim 1, wherein said metal nanoparticles have a diameter varying within the range of from 0.1 nm to 1000 nm.

5. The method of claim 1, wherein the final solution is prepared in a buffer solution having a pH comprised between 1 and 7.

6. The method of claim 1, wherein the color intensity of the final solution is detected with the naked eye.

7. The method of claim 1, wherein the color intensity of the final solution is detected by UV-visible spectroscopy.

8. The method of claim 7, wherein the color intensity of the final solution is detected by measuring absorbance at a wavelength between about 600 and 700 nm.

9. The method of claim 1, wherein the biological fluid comprises saliva, blood, sweat and urine.

10. The method of claim 1, wherein the food is a fruit juice or an oil.

11. A kit for determining antioxidant power of a sample of a biological fluid or a food, comprising a chromogenic peroxidase substrate and an aqueous solution of metal nanoparticles, wherein said metal nanoparticles comprise platinum optionally in combination with gold, palladium and/or silver.

12. The kit of claim 11 comprising:
a predetermined amount of an aqueous solution of metal nanoparticles, wherein said metal nanoparticles comprise platinum optionally in combination with gold, palladium and/or silver, at a concentration within the range of from 0.01 ppm to 1000 ppm;
a predetermined amount of a 3,3′,5,5′-tetramethylbenzidine (TMB) solution at a concentration within the range of from 0.001 M to 1 M; and optionally
a predetermined amount of hydrogen peroxide at a concentration comprised between 0.1 M and 10 M; and/or
a predetermined amount of an acetate buffer solution at a concentration comprised between 0.01 M and 1 M, having a pH value comprised between 1 and 7.

13. An in vitro diagnostic method for assessing oxidative stress in a subject, comprising determining antioxidant power of a biological fluid sample from the subject by the method of claim 1, wherein a decreased antioxidant power of the biological fluid sample from the subject compared to a reference sample or value is indicative of the oxidative stress of the subject.

14. The in vitro diagnostic method of claim 13, wherein the subject is suspected of conducting or conducts a health-damaging lifestyle, or the subject is suffering or is suspected to be suffering from a disease selected from the group comprising kidney damage, gout, endometriosis, diabetes and cancer.

15. The in vitro diagnostic method of claim 14, wherein the health-damaging lifestyle is alcohol abuse.

16. The in vitro diagnostic method of claim 14, wherein the health-damaging lifestyle is unhealthy diet.
